(19) United States
(12) Patent Application Publication
(10) Pub. No.: US 2023/0181446 A1
(43) Pub. Date: Jun. 15, 2023

(54) MOISTURIZING COMPOSITION AND METHOD

(71) Applicant: Owen Vincent, Minneapolis, MN (US)

(72) Inventor: Owen Vincent, Minneapolis, MN (US)

(21) Appl. No.: 17/644,169

(22) Filed: Dec. 14, 2021

(51) Int. Cl. A61K 8/73; A61K 8/92; A61K 8/06; A61K 8/34; A61Q 19/00

(52) U.S. Cl. CPC A61K 8/73; A61K 8/922; A61K 8/062; A61K 8/345; A61Q 19/007; A61K 2800/805

(57) ABSTRACT

A moisturizing solution and associated methods are disclosed. In one example, the moisturizing solution includes a moisturizing concentrate and an activation concentrate that are mixed together in water to form the moisturizing solution. In selected examples, the moisturizing concentrate and activation concentrate in water form a stable opaque cloud without separation of components.

### TECHNICAL FIELD

Embodiments described herein generally relate to moisturizing compositions and associated methods. Specific examples include a moisturizing concentrate and an activating solution.

### BACKGROUND

Moisturizers are often used to help soften skin and to aid in dry skin damage. It is desired to have moisturizers that can be suspended in water to better facilitate application of the moisturizers and to provide moisture along with the sealing properties of a moisturizer that helps trap moisture in the skin.

### BRIEF DESCRIPTION OF THE DRAWINGS

FIG. 1 shows selected steps in forming a moisturizing concentrate in accordance with some example embodiments.

FIG. 2 shows selected steps in forming an activation solution in accordance with some example embodiments.

FIG. 3 shows selected steps in forming a moisturizing solution in accordance with some example embodiments.

FIG. 4 shows a flow diagram of one example method of forming a moisturizing solution in accordance with some example embodiments.

### DESCRIPTION OF EMBODIMENTS

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

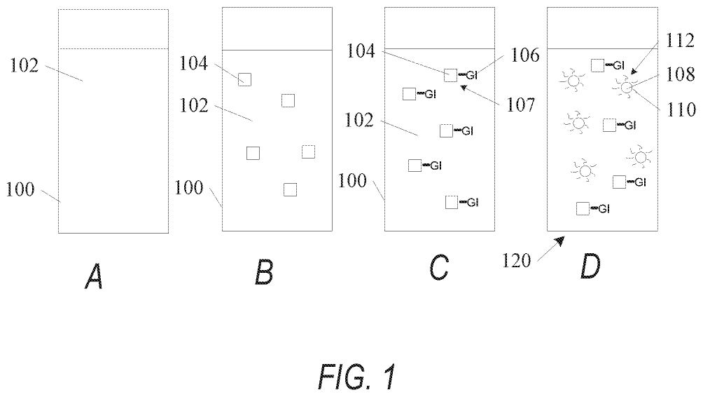

FIGS. 1A-ID show an example method of making a concentrated moisturizing solution. In FIG. 1A an amount of oil 102 is provided in a container 100. In one example, the oil includes mineral oil, although the invention is not so limited. Other oils 102 are also within the scope of the invention.

In FIG. 1B, an amount of xanthan gum 104 is added to the oil 102. By adding the xanthan gum to the oil, clumping of the xanthan gum is reduced or avoided. Xanthan gum may be used in an anhydrous powder form, and can clump in the presence of water or moisture. When the xanthan gum is immersed in oil, exposure to water is removed, and the xanthan gum will disperse in a substantially homogenous mixture.

In FIG. 1C, an amount of glycerol 106 is mixed with the xanthan gum 104. In the example shown, the glycerol 106 binds to the xanthan gum 104 and forms an emulsifier particle 107. The glycerol 106 will aid in later miscibility of the xanthan gum 104 with water as described in later operations.

In FIG. 1D, a surfactant 110 is added. In one example, the surfactant 110 emulsifies around a particle of oil 108 to form an emulsified oil particle 112. In one example, the surfactant 110 will aid in later miscibility of the particle of oil 108 with water as described in later operations. Examples of surfactant include, but are not limited to, Castille soap.

The solution in FIG. 1D forms a moisturizing concentrate 120 that can later be added to water to dilute to a desired strength. As noted above, the additions of glycerol and surfactant aid in the miscibility of the oil, and the xanthan gum when added to water to form an end user diluted strength solution.

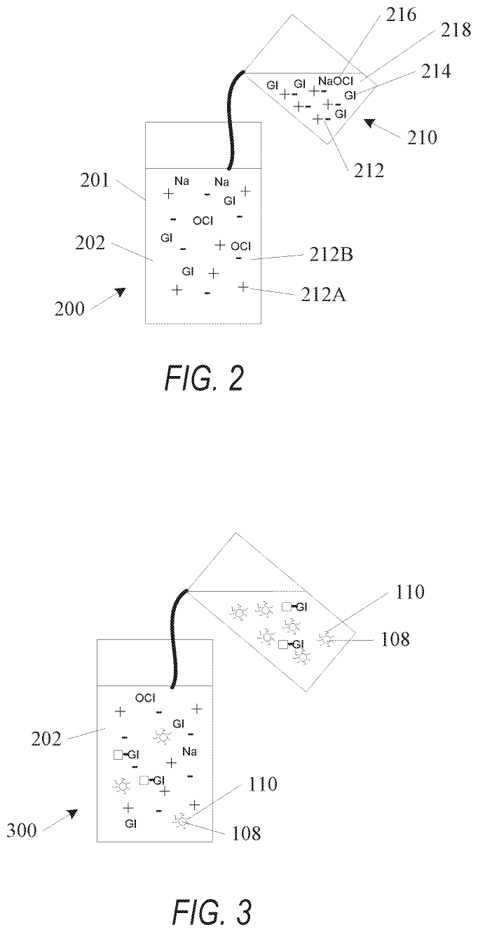

FIG. 2 shows a concentrated activating solution 210. In one example, the concentrated activating solution 210 includes a cationic salt 212. Examples of cationic salts include, but are not limited to, magnesium sulfate, sodium chloride, calcium chloride, calcium carbonate, magnesium carbonate, etc. The concentrated activating solution 210 further includes an amount of glycerol 214 and an amount of sodium hypochlorite 216. In the example shown, the active elements 214, 216 and 218 are included in a first amount of water 218 that forms the concentrated solution.

FIG. 2 further shows a final container 201 with a second amount of water 202. In practice, the concentrated activating solution 210 is added in a desired amount to the final container 201 and second amount of water 202 to form a diluted activating solution 200. As shown in FIG. 2, the diluted activating solution 200 includes positive ion 212A and negative ion 212B from the cationic salt 212. The sodium hypochlorite 216 is also shown in the final container 201 broken down into sodium ions and OCl ions.

In one specific example, the cationic salt 212 is magnesium sulfate. In one example, a concentration of the magnesium sulfate in the first amount of water 218 is between 16% w/v and 32% w/v in water solution. In a more specific example, a concentration of the magnesium sulfate in the first amount of water 218 is approximately 20% w/v. An amount of salinity in the concentrated activating solution 210 and in the final container 201 modifies a stability of a final moisturizing solution that is described in more detail below. In one example, a cationic salt concentration outside the 16% w/v to 32% w/v range is ineffective at stabilizing a moisturizing solution. In one example, a cationic salt concentration of approximately 20% w/v provides better stabilization of the moisturizing solution within the range 16% w/v to 32% w/v.

In one example, a concentration of the glycerol 214 in the first amount of water 218 is between 16% w/v and 32% w/v in the first amount of water 218. In a more specific example, a concentration of the glycerol 214 in the first amount of water 218 is approximately 20% w/v.

In one example, a concentration of the sodium hypochlorite 216 is between 0.10% and 1.00% in the first amount of water 218. In one example, a concentration of the sodium hypochlorite 216 is approximately 0.33% in the first amount of water 218. Selection of the amount of sodium hypochlorite 216 provides a number of advantages. One advantage includes reducing spoilage of the moisturizing solution. Another advantage includes increasing permeability in an end user's skin. In one example, a sodium hypochlorite 216 concentration outside the 0.10% to 1.00% range is ineffective at achieving these advantages. In one example, a sodium hypochlorite 216 concentration of approximately 0.33% provides better stabilization of the moisturizing solution within the range 0.10% to 1.00%.

In FIG. 3, the moisturizing concentrate 120 from FIG. 1D is added to the diluted activating solution 200 to form a moisturizing solution 300. As discussed above, the concentration of components such as the cationic salt 212, the glycerol 214, and the sodium hypochlorite 216 in the diluted activating solution 200 provide stability to the particles of oil 108. The moisturizing solution 300 forms an opaque cloud, less soluble without separation, that is very stable. An end user is able to shake up the moisturizing solution 300, leave the moisturizing solution 300 on a counter for days at room temperature, and the moisturizing solution 300 does not separate. The oil-surfactant emulsion is completely suspended in the water successfully and can moisturize with the oil particles 108 in an aqueous soak without a film or greasy after effect.

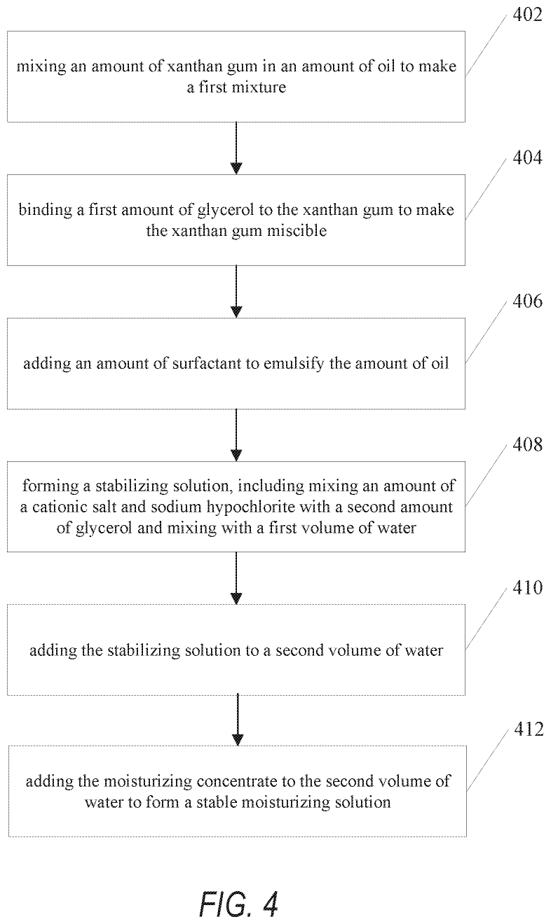

FIG. 4 shows one example method of forming a moisturizing solution, similar to the steps illustrated from FIGS. 1A-D and 2-3. In operation 402, an amount of xanthan gum is mixed in an amount of oil to make a first mixture. In operation 404, a first amount of glycerol binds to the xanthan gum to make the xanthan gum miscible. In operation 406, an amount of surfactant is added to emulsify the amount of oil. In operation 408, a stabilizing solution is formed. The stabilizing solution formation includes mixing an amount of magnesium sulfate and sodium hypochlorite with a second amount of glycerol and mixing with a first volume of water to form a concentrated solution. In operation 410, the stabilizing solution is added to a second volume of water. In operation 412, the moisturizing concentrate is added to the second volume of water to form a stable moisturizing solution.

To better illustrate the method and apparatuses disclosed herein, a non-limiting list of embodiments is provided here:

Example 1 includes a moisturizing solution. The moisturizing solution includes a moisturizing concentrate that includes an amount of an oil emulsified with a surfactant, and an amount of xanthan gum emulsified with a first amount of glycerol. The moisturizing solution includes a stabilizing solution. The stabilizing solution includes a cationic salt, and an amount of sodium hypochlorite, wherein cationic salt and the sodium hypochlorite are emulsified with a second amount of glycerol.

Example 2 includes the moisturizing solution of example 1, wherein the cationic salt is chosen from a group consisting of NaCl, CaCl, calcium carbonate, magnesium sulfate, and magnesium carbonate.

Example 3 includes the moisturizing solution of any one of examples 1-2, wherein the amount of an oil includes an amount of mineral oil.

Example 4 includes the moisturizing solution of any one of examples 1-3, wherein the surfactant includes castile soap.

Example 5 includes the moisturizing solution of any one of examples 1-4, wherein the magnesium sulfate is between 16% w/v and 32% w/v in water solution.

Example 6 includes the moisturizing solution of any one of examples 1-5, wherein the magnesium sulfate is approximately 20% w/v in water solution.

Example 7 includes the moisturizing solution of any one of examples 1-6, wherein the second amount of glycerol is between 16% w/v and 32% w/v in water solution.

Example 8 includes the moisturizing solution of any one of examples 1-7, wherein the second amount of glycerol is approximately 20% w/v in water solution.

Example 9 includes the moisturizing solution of any one of examples 1-8, wherein the sodium hypochlorite is approximately 0.33% in water solution.

Example 10 includes a method of forming a moisturizing solution. The method includes forming a moisturizing concentrate, including mixing an amount of xanthan gum in an amount of oil to make a first mixture, binding a first amount of glycerol to the xanthan gum to make the xanthan gum miscible, and adding an amount of surfactant to emulsify the amount of oil. The method includes forming a stabilizing solution, including mixing an amount of a cationic salt and sodium hypochlorite with a second amount of glycerol and mixing with a first volume of water to form a concentrated solution.

Example 11 includes the method of example 10, further including adding the stabilizing solution to a second volume of water, then adding the moisturizing concentrate to the second volume of water to form a stable moisturizing solution.

Example 12 includes the method of any one of examples 10-11, wherein mixing the amount of xanthan gum in the amount of oil includes mixing the amount of xanthan gum in an amount of mineral oil.

Example 13 includes the method of any one of examples 10-12, wherein adding the amount of surfactant to emulsify the amount of oil includes adding an amount of castile soap to emulsify the amount of mineral oil.

Example 14 includes the method of any one of examples 10-13, wherein mixing with the first volume of water includes mixing approximately 20% w/v magnesium sulfate and approximately 20% w/v glycerol.

Example 15 includes the method of any one of examples 10-14, wherein mixing with the first volume of water includes mixing sodium hypochlorite to approximately a 0.33% solution.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. A moisturizing solution, comprising:
a moisturizing concentrate, including;
an amount of an oil emulsified with a surfactant;
an amount of xanthan gum emulsified with a first amount of glycerol;
a stabilizing solution, including;
a cationic salt; and
an amount of sodium hypochlorite, wherein cationic salt and the sodium hypochlorite are emulsified with a second amount of glycerol.

2. The moisturizing solution of claim 1, wherein the cationic salt is chosen from a group consisting of NaCl, CaCl, calcium carbonate, magnesium sulfate, and magnesium carbonate.

3. The moisturizing solution of claim 1, wherein the amount of an oil includes an amount of mineral oil.

4. The moisturizing solution of claim 1, wherein the surfactant includes castile soap.

5. The moisturizing solution of claim 1, wherein the magnesium sulfate is between 16% w/v and 32% w/v in water solution.

6. The moisturizing solution of claim 1, wherein the magnesium sulfate is approximately 20% w/v in water solution.

7. The moisturizing solution of claim 1, wherein the second amount of glycerol is between 16% w/v and 32% w/v in water solution.

8. The moisturizing solution of claim 1, wherein the second amount of glycerol is approximately 20% w/v in water solution.

9. The moisturizing solution of claim 1, wherein the sodium hypochlorite is approximately 0.33% in water solution.

10. A method of forming a moisturizing solution, comprising:
forming a moisturizing concentrate, including;
mixing an amount of xanthan gum in an amount of oil to make a first mixture;
binding a first amount of glycerol to the xanthan gum to make the xanthan gum miscible;
adding an amount of surfactant to emulsify the amount of oil; and
forming a stabilizing solution, including mixing an amount of a cationic salt and sodium hypochlorite with a second amount of glycerol and mixing with a first volume of water to form a concentrated solution.

11. The method of claim 10, further including adding the stabilizing solution to a second volume of water, then adding the moisturizing concentrate to the second volume of water to form a stable moisturizing solution.

12. The method of claim 10, wherein mixing the amount of xanthan gum in the amount of oil includes mixing the amount of xanthan gum in an amount of mineral oil.

13. The method of claim 12, wherein adding the amount of surfactant to emulsify the amount of oil includes adding an amount of castile soap to emulsify the amount of mineral oil.

14. The method of claim 10, wherein mixing with the first volume of water includes mixing approximately 20% w/v magnesium sulfate and approximately 20% w/v glycerol.

15. The method of claim 14, wherein mixing with the first volume of water includes mixing sodium hypochlorite to approximately a 0.33% solution.
